(19) United States
(12) Patent Application Publication
(10) Pub. No.: US 2021/0005326 A1
(43) Pub. Date: Jan. 7, 2021

(54) CUSTOMIZABLE COMMUNICATION PLATFORM WITH ADJUSTABLE GUARDRAILS

(71) Applicant: CAREMINDR Corporation, Los Gatos, CA (US)

(72) Inventors: Harry Raymond Soza, San Jose, CA (US); Irina Yermilov, Los Angeles, CA (US)

(21) Appl. No.: 16/919,021

(22) Filed: Jul. 1, 2020

Related U.S. Application Data
(60) Provisional application No. 62/869,483, filed on Jul. 1, 2019.
(60) Provisional application No. 62/869,497, filed on Jul. 1, 2019.

(51) Int. Cl. G16H 50/70; G16H 10/20; G16H 70/20; G16H 50/20; G16H 50/30; G16H 40/20

(52) U.S. Cl. CPC G16H 50/70; G16H 10/20; G16H 40/20; G16H 50/20; G16H 50/30; G16H 70/20

(57) ABSTRACT

Processing patient information and other treatment plan information may require access to a patient profile and other third parties involved in the patient treatment plan. One example method includes linking a patient device and a healthcare provider server, requesting an at least one patient response of at least one query of an at least one health related issue, receiving at least one patient response, receiving at least one guardrail value of the at least one patient response, triggering an approach alarm if it is determined that a proximity of an at least one historical trend approaches the at least one guardrail value, triggering a crossover alarm if it is determined that the at least one patient response crosses over the at least one guardrail value, providing the request, the at least one patient response and the at least one guardrail value to a healthcare provider device.

### TECHNICAL FIELD

This application relates to a customizable communication platform and more particularly to providing customized healthcare communication to a user device by integrating various personal records with an ongoing communication regiment, wherein the communication includes guardrail alerts for a healthcare provider device.

### BACKGROUND

Conventionally, the approach to providing users with ongoing communications regarding a plan or other repetitive course of action may leave the majority of the work to the user. The smartphone and other personal computing devices are not being properly utilized when offering users with options for maintaining a course of treatment or a set of goals. The lack of action taken by the professional service provider and/or the user can lead to personal health problems and lost revenue for providers, insurers, etc., as well as the users.

### SUMMARY

Example embodiments of the present application provide a first example method of the present application including linking a patient device and a healthcare provider server, requesting by a cloud server an at least one patient response to an at least one message sent to the patient device including an at least one query of an at least one health related issue, receiving by the healthcare provider server from the patient device the at least one patient response to the at least one query, receiving from the cloud server an at least one guardrail value of the at least one patient response, triggering an approach alarm if it is determined that a proximity of an at least one historical trend of the at least one patient response approaches the at least one guardrail value, triggering a crossover alarm if it is determined that the at least one patient response crosses over the at least one guardrail value, providing the request, the at least one patient response and the at least one guardrail value to a healthcare provider device if the at least one patient response triggers at least one of the approach alarm and the crossover alarm, determining a set of possible reasons that the at least one patient response has triggered at least one of the approach alarm and the crossover alarm, if triggered, and presenting to the healthcare provider device a list of alternative treatment regimens in response to the determined set of possible reasons if the at least one patient response triggers at least one of the approach alarm and the crossover alarm.

A second example embodiment of the present application provides a non-transitory computer readable medium comprising instructions that, when read by a processor, cause the processor to perform requesting by a cloud server an at least one patient response to an at least one message sent to a patient device including an at least one query of an at least one health related issue, receiving by a healthcare provider server the at least one patient response from the patient device to the at least one query, receiving from the cloud server an at least one guardrail value of the at least one patient response, triggering an approach alarm if it is determined that a proximity of an at least one historical trend of the at least one patient response approaches the at least one guardrail value, triggering a crossover alarm if it is determined that the at least one patient response crosses over the at least one guardrail value, providing the request, the at least one patient response and the at least one guardrail value to a healthcare provider device if the at least one patient response triggers at least one of the approach alarm and the crossover alarm, determining a set of possible reasons that the at least one patient response triggered at least one of the approach alarm and the crossover alarm, if triggered and presenting to the healthcare provider device a list of alternative treatment regimens in response to the determined set of possible reasons if the at least one patient response triggers at least one of the approach alarm and the crossover alarm.

A further example embodiment of the present application provides a system, comprising an at least one cloud-based processor, and at least one memory electrically coupled to the at least one cloud-based processor and storing an application, wherein the at least one cloud-based processor performs operations to request by a cloud server an at least one patient response to an at least one message sent to a patient device including an at least one query of an at least one health related issue, receive by a healthcare provider server the at least one patient response from the patient device to the at least one query, receive from the cloud server an at least one guardrail value of the at least one patient response, trigger an approach alarm if it is determined that a proximity of an at least one historical trend of the at least one patient response approaches the at least one guardrail value, trigger a crossover alarm if it is determined that the at least one patient response crosses over the at least one guardrail value, provide by the cloud server, the request, the at least one patient response and the at least one guardrail value from the cloud server to a healthcare provider device if the at least one patient response triggers at least one of the approach alarm and the crossover alarm, determine a set of possible reasons that the at least one patient response triggered at least one of the approach alarm and the crossover alarm, if triggered and present to the healthcare provider device a list of alternative treatment regimens in response to the determined set of possible reasons if the at least one patient response triggers at least one of the approach alarm and the crossover alarm.

Another example embodiment of the present application including linking a patient device and a healthcare provider server, requesting by a cloud server an at least one patient response to an at least one message sent to the patient device including an at least one query of an at least one health related issue, receiving by the healthcare provider server the at least one patient response from the patient device to the at least one query, receiving from the cloud server an at least one low urgency threshold value of the at least one patient response, triggering a failure to progress alarm if it is determined that a historical trend of the at least one patient response indicates a failure to progress trend, providing the request, the failure to progress trend and the at least one low urgency threshold value to a healthcare provider device if the failure to progress alarm is triggered, determining a set of possible reasons that the at least one patient response triggered the failure to progress alarm, if triggered and presenting to the healthcare provider device a list of alternative treatment regimens in response to the determined set of possible reasons if the at least one patient response triggered the failure to progress alarm.

### BRIEF DESCRIPTION OF THE DRAWINGS

FIG. 1 illustrates an example of the integrated application platform according to example embodiments.

FIG. 2 illustrates a network configuration of the third party participants of the integrated application according to example embodiments.

FIG. 3 illustrates a user smartphone interface of an example treatment plan according to example embodiments.

FIG. 4A illustrates an example setup configuration for a new course of treatment according to example embodiments.

FIG. 4B illustrates an example database entry for the new course of treatment according to example embodiments.

FIG. 4C illustrates a flow diagram configuration for the new course of treatment according to example embodiments.

FIG. 4D illustrates an example list of messages for the ongoing course of treatment according to example embodiments.

FIG. 4E illustrates an example setup configuration for various courses of treatment according to example embodiments.

FIG. 4F illustrates an example set of details of an ongoing course of treatment according to example embodiments.

FIG. 4G illustrates an example network configuration of the various third parties involved in the application operation and compliance according to example embodiments.

FIG. 5 illustrates a logic module configured to process the input and output parameters of the application according to example embodiments.

FIG. 6 illustrates an example network entity device configured to store instructions, software, and corresponding hardware for executing the same, according to example embodiments of the present application.

FIG. 7 illustrates a data file defining tags and levels of urgency, according to example embodiments of the present application.

FIG. 8 illustrates a patient reply, according to example embodiments of the present application.

FIG. 9 illustrates an electronic report to a healthcare provider device with alert tags, according to example embodiments of the present application.

FIG. 10 illustrates system architecture, according to example embodiments of the present application.

FIG. 11 illustrates a first method, according to example embodiments of the present application.

FIG. 12 illustrates a second method, according to example embodiments of the present application.

FIG. 13 illustrates a first non-transitory computer readable medium, according to example embodiments of the present application.

FIG. 14 illustrates a third method, according to example embodiments of the present application.

FIG. 15 illustrates an example programming input for guardrails warning and alert levels, according to example embodiments of the present application.

FIG. 16 illustrates an example patient data chart, according to example embodiments of the present application.

FIG. 17 illustrates an example failure to progress chart for systolic blood pressure, according to example embodiments of the present application.

FIG. 18 illustrates a fourth method, according to example embodiments of the present application.

### DETAILED DESCRIPTION

It will be readily understood that the components of the present application, as generally described and illustrated in the figures herein, may be arranged and designed in a wide variety of different configurations. Thus, the following detailed description of the embodiments of a method, apparatus, and system, as represented in the attached figures, is not intended to limit the scope of the application as claimed, but is merely representative of selected embodiments of the application.

The features, structures, or characteristics of the application described throughout this specification may be combined in any suitable manner in one or more embodiments. For example, the usage of the phrases “example embodiments”, “some embodiments”, or other similar language, throughout this specification refers to the fact that a particular feature, structure, or characteristic described in connection with the embodiment may be included in at least one embodiment of the present application. Thus, appearances of the phrases “example embodiments”, “in some embodiments”, “in other embodiments”, or other similar language, throughout this specification do not necessarily all refer to the same group of embodiments, and the described features, structures, or characteristics may be combined in any suitable manner in one or more embodiments.

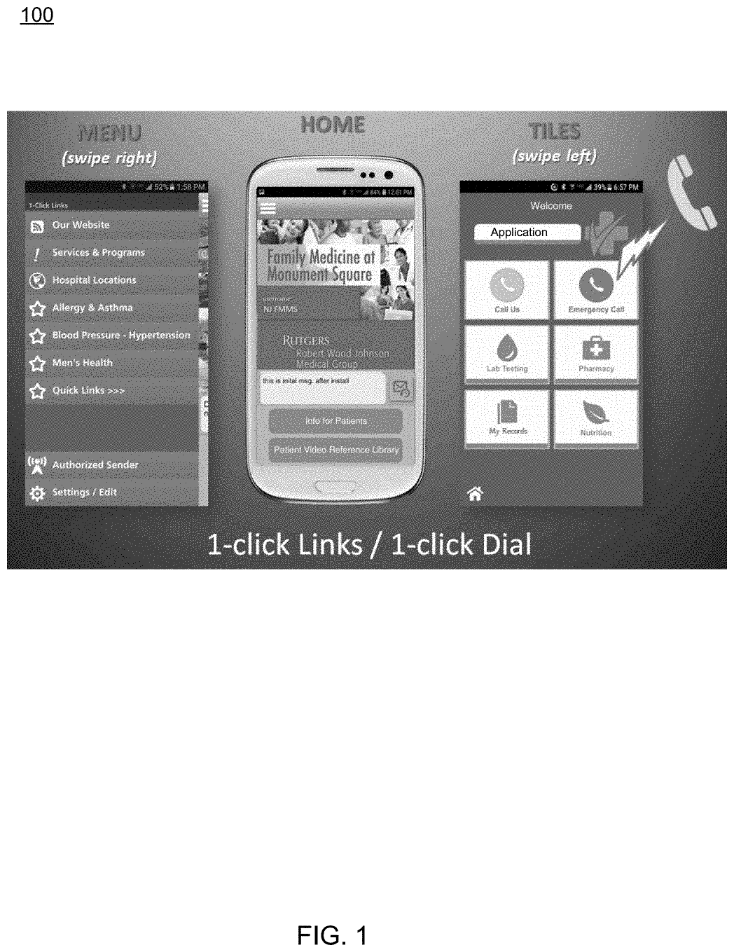

FIG. 1 illustrates an example of the integrated application platform according to example embodiments. Referring to FIG. 1, the configuration 100 includes a menu user interface, a home user interface and a set of option tiles for accessing third party resources, such as test results, emergency concerns, pharmacy information, etc. The tiles may initially be provided by the cloud system and may serve as a conduit of previous learning pertaining to a specific user's condition, treatment plan and schedule. The tiles may be added to by healthcare professionals, in which case the cloud system will include the additional information to perform additional linkages between conditions, treatment plans, drugs, timings and the like. The tiles have within them not just the specific item, but the interlinkages that make insight into specific outcomes much more insightful and make the computer outputs much more targeted.

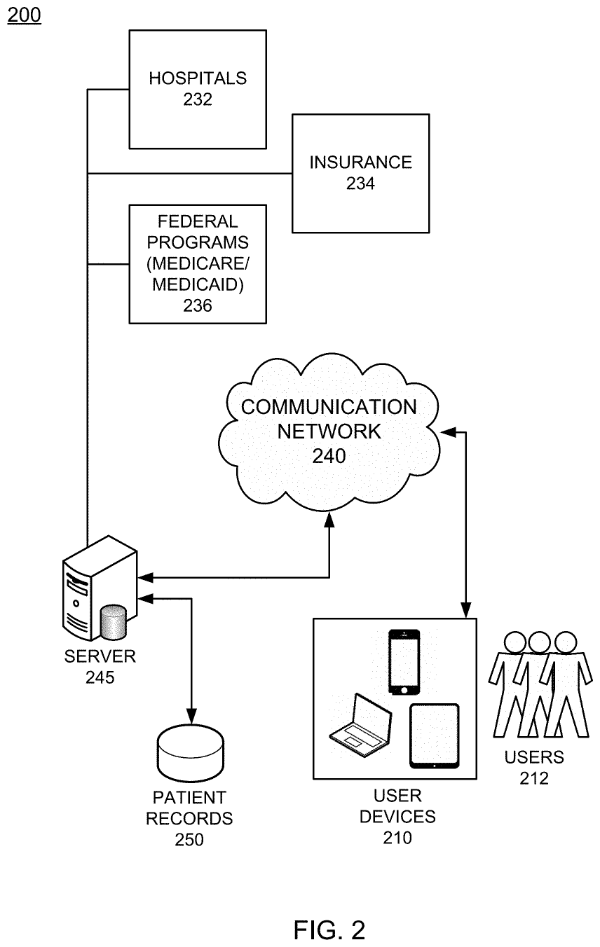

FIG. 2 illustrates a network configuration of the third party participants of the integrated application according to example embodiments. Referring to FIG. 2, the network includes a central server 245 with patient records 250. The information needed to provide treatment plans and other integrated services may require access to hospital and other provider services 232, insurance company information 234, drug providers, federal program administrators 236, etc. The information may be incorporated into any treatment plan or other integrated service model accessed by a user device 210 operated by a user 212. The servers and third party modules may operate on-site or in a cloud network managed by the providers.

Examples of treatment plans and other objectives may include a care management service for assessment of patient medical needs. The system and application may ensure timely receipt of all recommended treatment actions, drugs, third party services over a designated period of time. Also, referrals to other providers and additional services may provide emergency visits, discharge instructions, nursing facility operations, and home healthcare functions. In operation, the procedure may begin with the medical treatment provider creating a treatment plan or ‘journey’ for each patient. Each journey is generally for a single chronic condition or objective. One patient may have multiple journeys integrated into a single application. Also, the journeys may originate from various different providers and service entities. The journey will provide the healthcare provider device with biometric, objective and subjective data to enable evidence-based medical decisions. As an example, the biometric data may be glucometer data collected from a blue tooth enabled device and made available to the physician, objective data such as whether the patient visited an emergency room or hospital and subjective data such as how the patient is feeling.

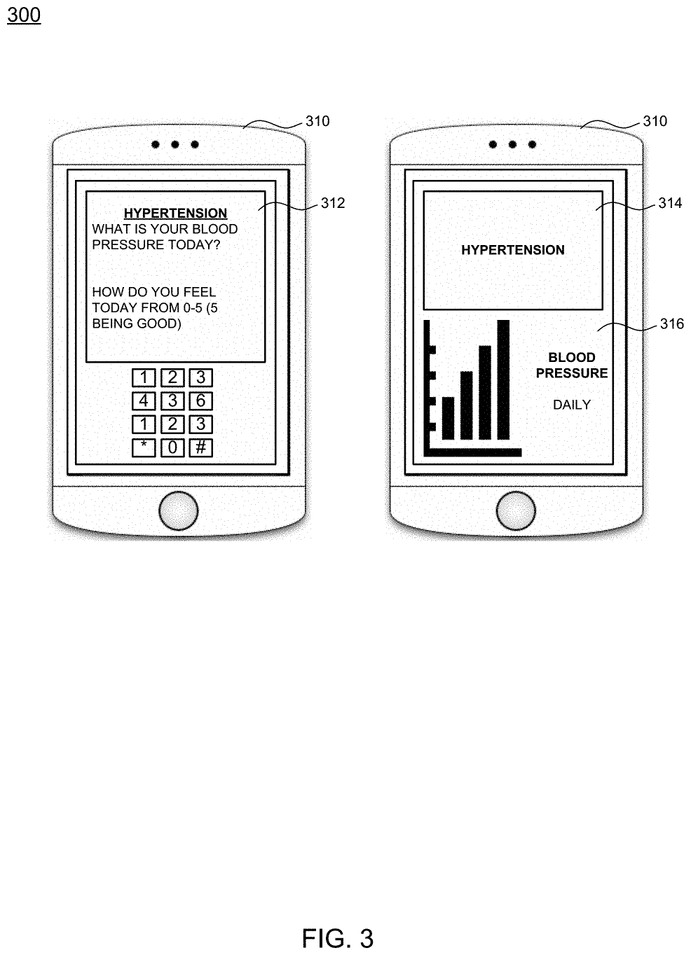

FIG. 3 illustrates a user smartphone interface of an example treatment plan according to example embodiments. Referring to FIG. 3, the journey for “hypertension” may have been created or modified by a patient doctor and may include an interface 300 with a smartphone device 310 and a screen option configuration providing questions 312, information about the treatment, reminders and other functions. The example in FIG. 3 provides for a set of questions 312 and a journey topic 314 along with a graph of blood pressure records 316 as measured over time from various interactions. The system has the capability to connect the application on the patient device to a machine, via a wireless connection such as Bluetooth, such that it may provide medical information directly to the application. Utilizing this capability, the patent device may receive a current reading such as temperature, blood pressure, heart rate, weight, etc., and have that information dynamically provided to the application which may use that information to provide immediate feedback and recommendations.

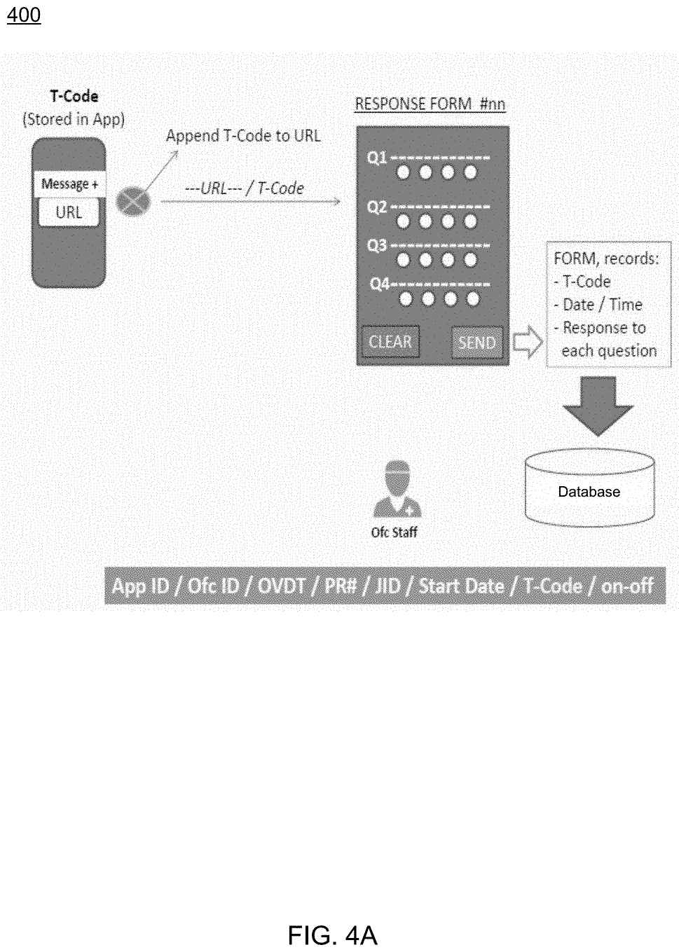

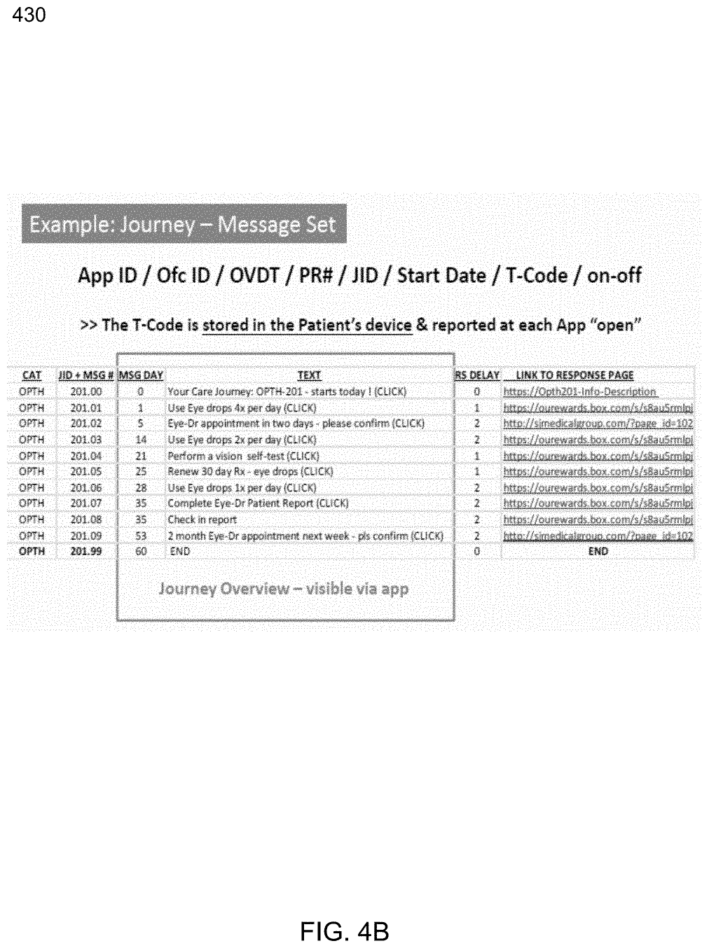

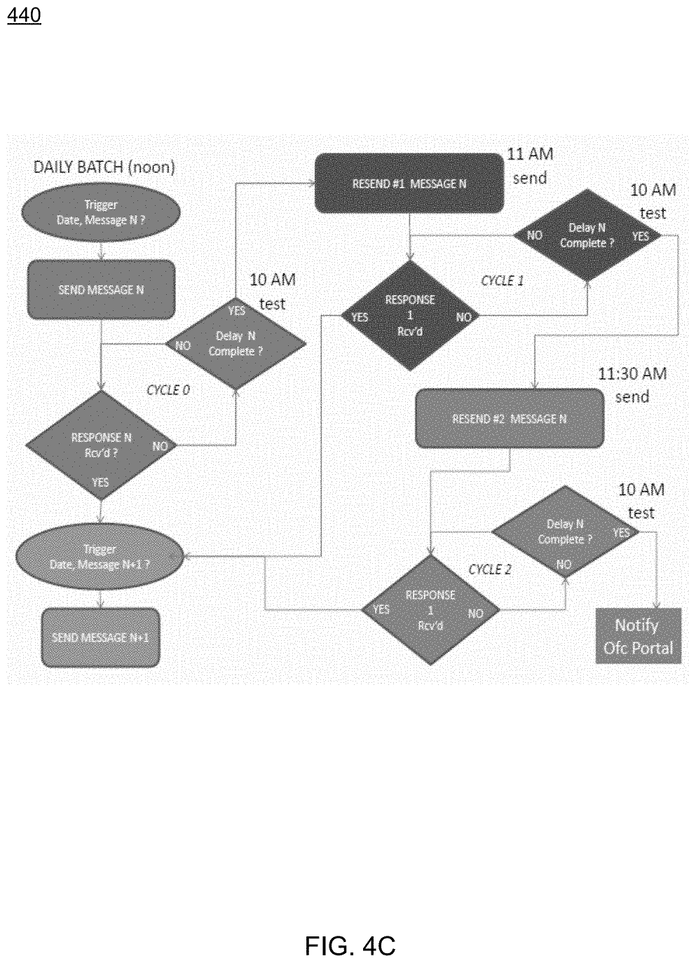

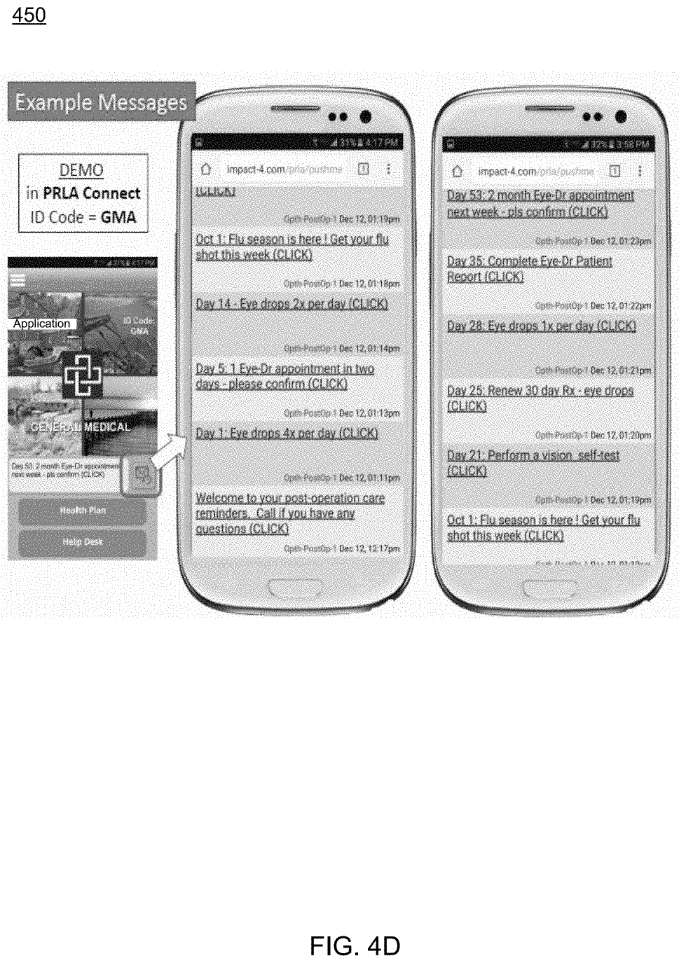

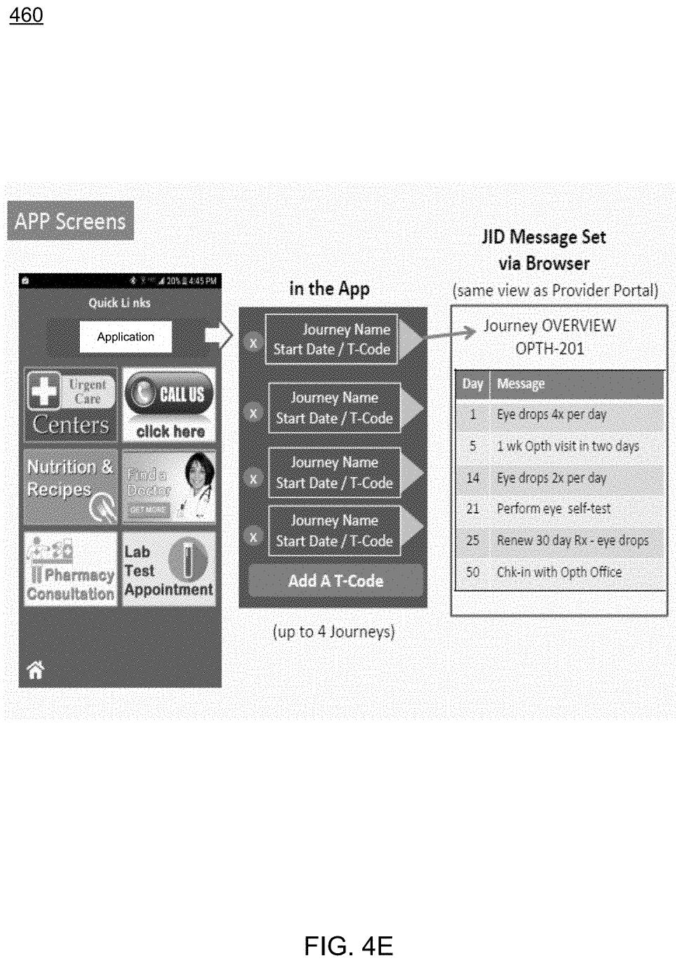

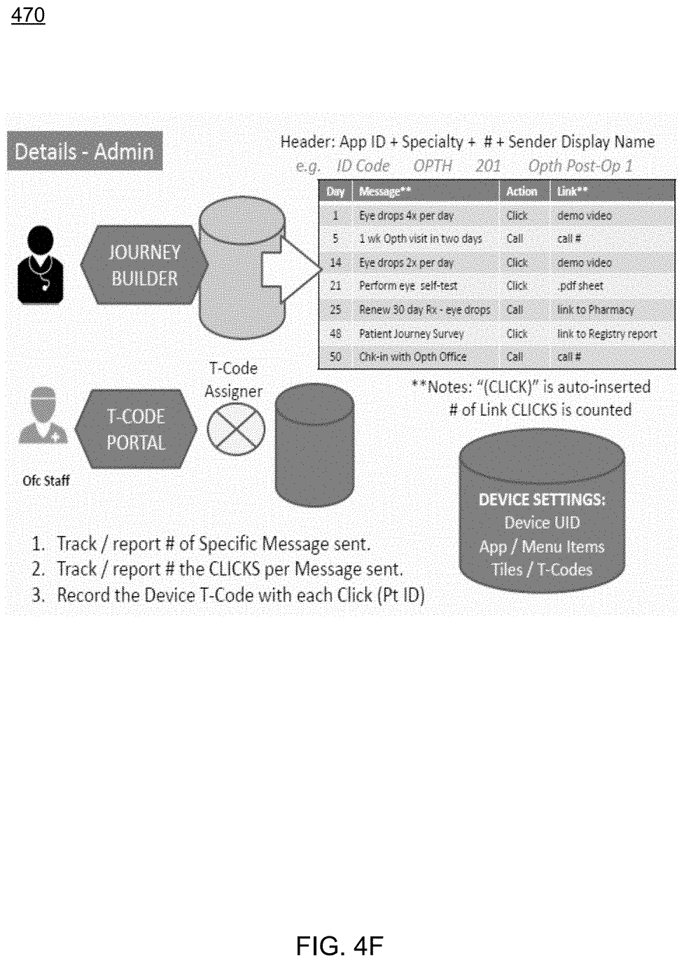

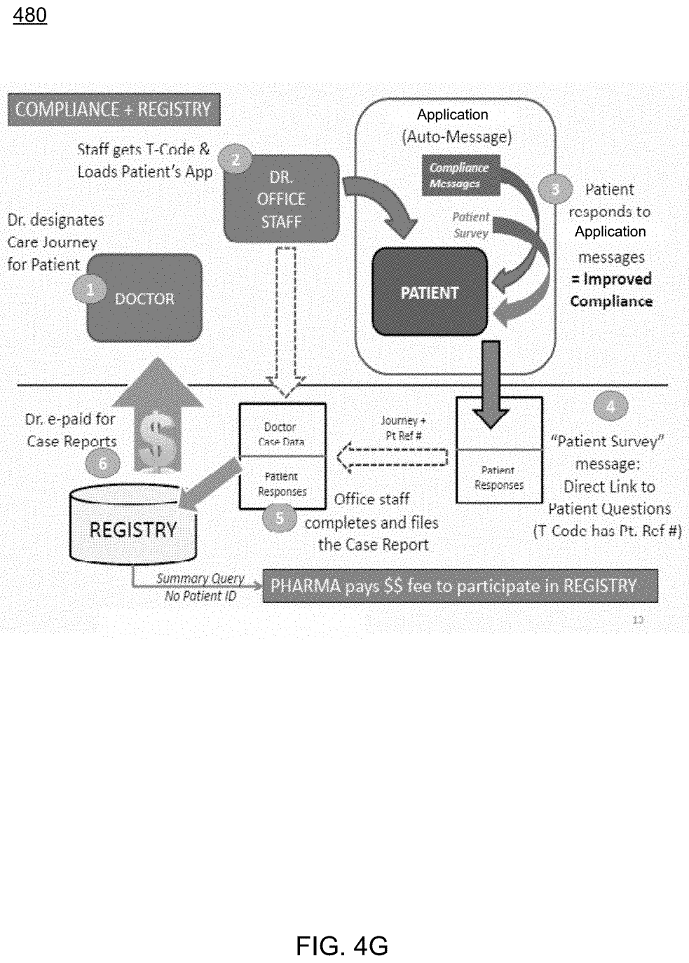

FIG. 4A illustrates an example setup configuration for a new course of treatment according to example embodiments. Referring to FIG. 4A, the illustration 400 includes the basic setup functions of linking a particular journey T-code (unique code) to the message and/or universal resource locator (URL) to link the application of the user device to a customized template, such as a response form, questionnaire, etc. The unique T-code, date, time, response, and other records for each instance may be stored in a patient record managed by the application system database. A T-code means return to provider (RTP) which indicates that a claim has reached its final disposition with no reimbursement due to billing errors. At the bottom of 4A an App ID is the applicant identification, the OVDT is the office visit date and time, PR# is the patient reply number and JID is the journey identification number. The database uses these identifiers to identify patients and isolate which forms to utilize. The form is dynamically filled out in the cloud server 245 with information from the patient records 250.

FIG. 4B illustrates an example database entry for the new course of treatment according to example embodiments. Referring to FIG. 4B, the example configuration 430 includes a database entry of messages which are organized by a category, in this case ophthalmology, and with a message content, including a link to a response page. The context and add-ons of a particular message may be customized based on a preferred layout or a default layout. The messages may be categorized in multiple ways by the cloud server, by patient, by journey, by response, by treatment, by drug, by drug interaction, and the like, in this way interactions of various kinds may be grouped by the system to potentially find commonalities, similarities, beneficial effects, side effects, etc. The response page may be similarly chosen by the cloud server based upon the initial journey or one of the groupings. The search by the cloud based server for similarities and interactions may allow the system to continuously learn from the patients what is the most favorable treatment and what common issues arrive and how those issues are dealt with for other patients having similar demographics and responses. The healthcare professional may also customize the interaction with the patient and the response of the system to certain criteria which he indicates.

FIG. 4C illustrates a flow diagram 440 configuration for the new course of treatment according to example embodiments. Referring to FIG. 4C, the flow diagram includes a daily batch of messages which are set up to be delivered to one or more devices associated with one or more assigned patients. The process begins with a trigger to send a message, such as a matured date or time. The process then continues to deliver additional messages once confirmation of delivery is made. If the message is delayed or the response required is not received, the message may be resent as a late message requiring immediate attention. The process may continue to cycle to identify whether any messages are outstanding or have not been confirmed. The system may track information here related to the type of illness/issue, treatment program, monitoring/effectiveness of a patient and tag according to responses and non-responses of the patient. The system may change tactics if the patient is unresponsive or answers responses indicating issues associated with the treatment journey. The system may change which questions are asked or the frequency of messaging based on the patient response or non-response. In the situation in which a patient is not responding, the system may have the healthcare provider initiate a call directly to the patient.

FIG. 4D illustrates an example list of messages for the ongoing course of treatment according to example embodiments. Referring to FIG. 4D, in this illustration 450, the various messages intended for a particular patient are illustrated by date. The messages may originate at the cloud based system and may be originally designed into the specific journey. The timing and response criteria may be set by the journey and may be modified by the healthcare professional.

FIG. 4E illustrates an example setup configuration for various courses of treatment according to example embodiments. Referring to FIG. 4E, the configuration 460 includes a menu of options along with a set of potential journeys the user device may be assigned to manage the ongoing healthcare treatment plans. The overview of treatment options and dates are included for reference purposes. The patient may be interacting with the system for multiple journeys simultaneously. Each journey may interact with the patient according to its own logical construct. The messages to be sent may originate at the cloud based server and the data received may be received by both the cloud based server and the healthcare provider server. In the cloud server the data is sorted according to patient demographics and interactions, in the healthcare provider server the data is sorted according to patient and healthcare provider. The logic governing the interactions may be controlled by the cloud based server and may be initially set by the specific journey.

FIG. 4F illustrates an example set of details of an ongoing course of treatment according to example embodiments. Referring to FIG. 4F, the details of the administrator are shown to include a journey builder function based on certain parameters, such as an identification code, specialty, a number and a sender name. The number of messages, responses and actions are recorded to demonstrate interaction with the application and the specific treatment plan(s). The journey builder utilizes a spreadsheet style input that has a day, the message to send to the patient, the expected response and a link for the patient to link to. The system tracks the interactions between the sent messages and the patient responses. The patient device is tracked by a device universal identification (UID) and the application and menu items, the selection tiles and the T-codes. The system tracks and reports the number of specific messages sent to the patient, the number of clicks the patient performs and records the device T-code with each click in association with the patient identification number (Pt ID).

FIG. 4G illustrates an example network configuration of the various third parties involved in the application operation and compliance according to example embodiments. Referring to FIG. 4G, the network of communications among the integrated platform 480 demonstrates the process initiating with the doctor's office establishing a journey for the patient and assigning a T-code (unique code). The patient's responses are identified along with links and references to third party message links and other information sources. The doctor designates a journey for the patient based on the patient's condition and the treatment plan. The healthcare providers staff gets a T-code for the treatment plan and sends the application to the patients device. The journey application sends messages to the patient, receives responses to surveys and assesses compliance to the journey protocols. The patient responses to the patent survey messages directly link the response to the query for that patient. The journey sends the data to the cloud server in scrubbed form and sends the data to the healthcare providers server linking the patient reference number to the journey. The doctor's case files and the patient responses are compiled into a case report that is sent to a registry.

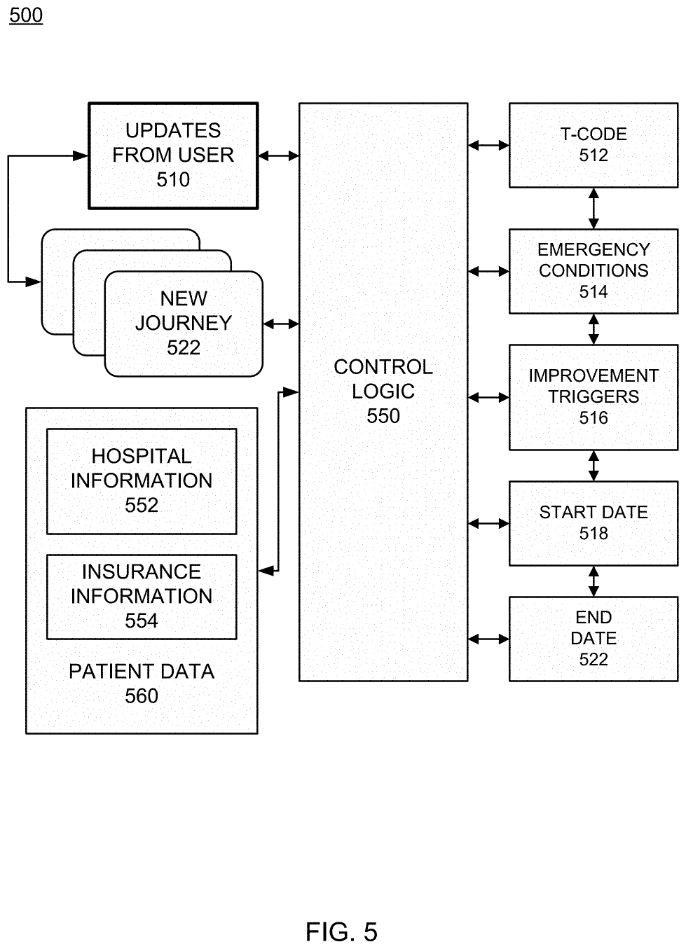

FIG. 5 illustrates a logic module configured to process the input and output parameters of the instant application according to example embodiments. Referring to FIG. 5, the control logic platform 500 includes a control logic unit 550, such as a processor or other processing entity that may receive updates from a user 510 such as by keyboard, voice processing and the like, new journey information 522 and/or patient data 560 including hospital 552, insurance 554, doctor, office, prescriptions and the like. The logic may be configured to identify and link the unique T-code 512, emergency conditions 514, improvement triggers 516 for optimal changes to the treatment plan, along with dates 518 and new journey information 522 to perform the treatment plan.

In addition, while the term “message” has been used in the description of embodiments of the present application, the application may be applied to many types of network data, such as, packet, frame, datagram, etc. For purposes of this application, the term “message” also includes packet, frame, datagram, and any equivalents thereof. Furthermore, while certain types of messages and signaling are depicted in exemplary embodiments of the application, the application is not limited to a certain type of message, and the application is not limited to a certain type of signaling.

According to example embodiments, a user device, such as a smartphone, cellular phone, tablet device, laptop or other computing device with a memory and processor, may communicate with another computing device and/or a server to provide an integrated communication platform.

Example embodiments provide a computer system programmed to use automated messaging from medical offices to specific patients. The application is not limited to medical procedures and functions and may be used with other configurations for various purposes and services benefitting the end user. Example embodiments include three main computer systems, which work together in an integrated manner including a management platform that controls set-up, functionality, activity reporting, and messaging credentials for the users. An administrative platform which the doctor and doctor's office can access via the internet, and a mobile application that a patient can download into a mobile computer device such as a smartphone or tablet. The management platform acts as the nexus of the system sending outgoing messages on behalf of the healthcare provider and forwarding patient responses to the healthcare provider's administrative platform. The medical office may have a specific identification that is stored within the management platform.

The integrated platform provides a way of checking-in with a patient at prescribed intervals during times between office visits and when undergoing certain treatment that the doctor is providing or overseeing for the patient. The patient dialog may gather relevant information about the status of the patient's conditions or recovery and can be modified or tailored to specifically meet the dialog requirements of the treating physician. Once initiated by the doctor's office, the application operates in an autonomous manner by delivering messages to the patient to prompt responses if needed. The application functions are monitored to assure that the patient replies to the information requests from the doctor, otherwise a no-response alert is sent to the doctor's office. The interactions are recorded and time-stamped, providing an auditable record of the dialog, suitable for insurance billing purposes. The application can also support biometric information from devices that measure certain body functions, such as diabetes glucometers, or blood pressure cuffs, or any sensory readable healthcare metric. The application may also create a longitudinal record of information for the patient to illustrate week-to-week trends.

Response Alert Tags

As has been stated earlier, this method and system is utilized when a patient visits a healthcare provider for an illness/condition which is diagnosed and treated. The treatment occurs over a period of time and is referred to as a journey. The system tracks a patient's progress along the journey for that illness or condition and solicits health information from the patient at clinically-relevant intervals, across an extended time period to enable evidence based medicine. The specific information sought, the intervals, and the time period duration apply to specific conditions or illnesses for which a specific patient is being treated.

This solicitation for patient information from the cloud server may take the form of queries sent to the patient for information, when the responses to those queries are delivered to the patient's healthcare provider device (e.g. physician). The patient's journey may have a number of waypoints occurring at the clinically-relevant intervals. The responses to the queries at these waypoints are meant to determine a patient's progress and status and to present to the healthcare provider device evidence upon which to conduct evidence-based medicine. The responses are collected by the system and measured against historical norms for the patient and/or expected answers for similar patients on similar journeys.

In the event of an unexpected response to a query at that waypoint, the response is treated as notable. Notable events may be considered non-urgent or may be considered urgent or emergent. This divergence from the expected response outcome is graded for severity or urgency. If the severity or urgency of the response exceeds a predetermined threshold for that patient for that journey for that illness or condition at that waypoint, an urgent tag is created and sent to the healthcare provider device. The grading may be one of an immediate medical action advisory, a follow-up advisory and a medical history review advisory.

The information requested in the query is sent in a structured format to allow ease in answering and the response data is delivered to the healthcare provider device in a structured data format to facilitate ease of analysis and trend detection.

The response alert tag is a feature that “tags” certain responses provided by the patient as information that requires follow-up or special notice by the patient's healthcare provider. The tag may indicate a level of severity or urgency, thus alerting the provider to information that may need immediate medical action, additional follow-up with the patient or a specific review of the patient's medical history. The tag settings are set at the cloud server and the alert tags are sent by the cloud server.

The tag may be communicated to the provider through multiple channels depending upon circumstance and urgency and in an immediate manner or in a weekly aggregated format depending in part upon urgency.

Work flow instructions may be electronically linked to a tag, so that the specific healthcare provider that reviews the data will have guidance about the actions to be taken when a tag appears and any escalation of clinical review that might be appropriate.

Each patient for each illness or condition is interacted with by the system at intervals which are relevant to that illness or condition and the queries are sent to determine the patient's progress or status. The received response to the query is measured against an expected response, and anomalies or offsets are noted. If these response anomalies or offsets are larger than a predetermined amount, an urgent or severe issue may need to be addressed. Thus the response is tagged as urgent and may be sent utilizing a priority delivery schedule, a priority delivery indicia on the response and may be made to a priority delivery list determined by the healthcare provider. The response may be tagged as non-urgent if the determined urgency level does not meet the predetermined urgency threshold of the patient for the health related issue.

The structured format allows an overlap of queries so that the patient is not answering multiple identical queries at any one point in time. Additionally, the structured format allows the data to be collected and logged in a structured format and assembled for future review both by the practitioner and the patient to determine trends.

In one example, a method includes requesting via a cloud based system from a patient response to a query and receiving the response to the health related query, determining an urgency level of the response by the cloud based server, based on the patient health related issue and tagging by the cloud based server the response as urgent if the determined urgency level exceeds a predetermined urgency threshold of the patient for the health related issue.

The method may also include providing the urgent tagged response to the health provider by the cloud based server, where the urgent tagged response may be sent from the cloud based server utilizing a priority delivery schedule, a priority delivery indicia for the response and may be made to a priority delivery list. The output of the tagged responses may also be prioritized by urgency.

The method may also include tagging by the cloud based server the response as non-urgent if the determined urgency level does not meet the predetermined urgency threshold of the patient for the health related issue.

If the determined urgency level of the response is such that it rises to the level of a medical emergency, then the primary care physician may be immediately notified as well as emergency services such as 911 and if deemed appropriate, dispatched to the location identified either by the patient or gathered from a location indicator in his mobile device. The urgency level determination may be performed by the cloud based server. If the response is deemed critical, in situations where the primary physician is not immediately available, an emergency medical specialist may be placed in active direct communication with the patient. The system would make available to the first responder the query and response to provide context for the escalation.

The response may be graded as to the tagged urgency level of the response, where the grading is at least one of an immediate medical action advisory, a follow-up advisory and a medical history review advisory. A follow-on query may be sent based on the urgent tagged response to give the provider context to the urgent tagged response. As an example, if the patient responds that they have been to the emergency room (ER) that may trigger another set of queries about the ER visit to add context to the response. This second set of queries may determine whether the ER visit was related to conditions or illnesses related to the journey, or whether visit was for a condition unrelated to the journey, but still of interest to the healthcare provider.

In another example a cloud based system links a patient device and a healthcare provider server. The cloud based system requests a response to a query from a patient pertaining to a health related issue, receives the response to the query and determines an urgency level of the response based on the patient health related issue. The system also tags the response as urgent if the determined urgency level exceeds a predetermined urgency threshold of the patient for the health related issue and provides the urgent tagged response to health provider.

The cloud based system may receive via the patient device a sensor signal provided by a medical device in response to the query. The medical device may be a blood pressure monitor, a glucometer, a pulse meter, a continuous positive airway pressure device, a heart monitor, an implanted medical device and the like.

The cloud based system may receive via the patient device an audio or text message indicating a medical distress condition in response to the query or may overhear the patient indicating a medical distress condition in conversations or texts in an unsolicited message.

The system may also interpret patient actions in regards to patient historical norms, such as, if the patient is overheard slurring his speech, he may be having a stroke, or if he is discussing that he has pressure in his chest or his left arm is numb, he may be having a heart attack. At this point the system may connect him directly to a medical specialist and take other appropriate action, such as determining his location and dispatching emergency services.

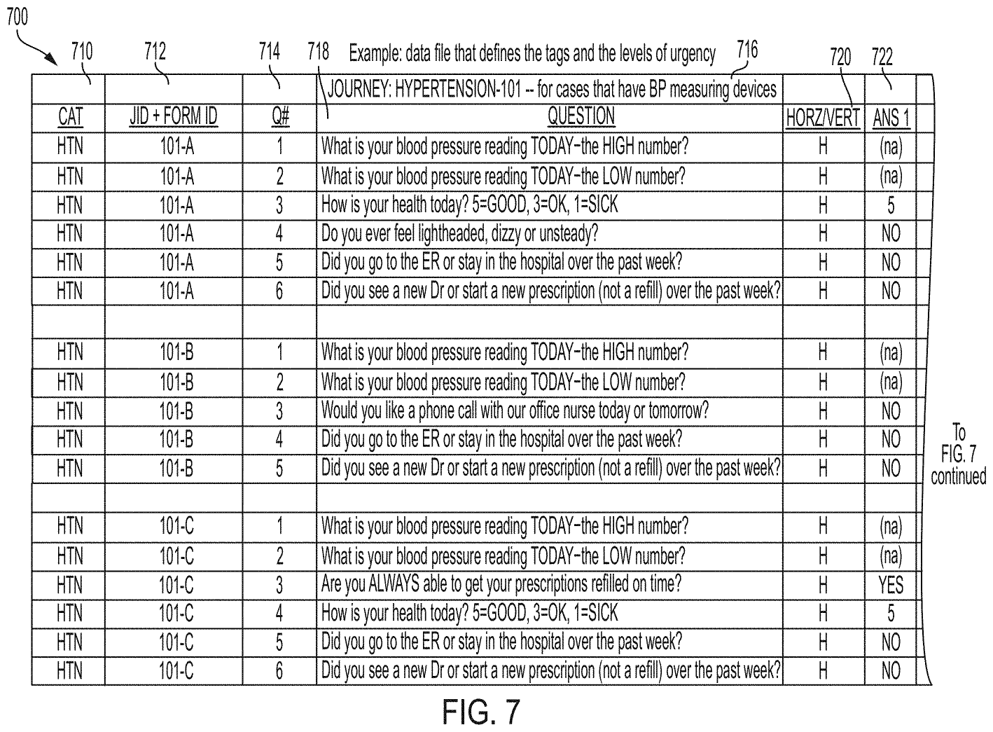

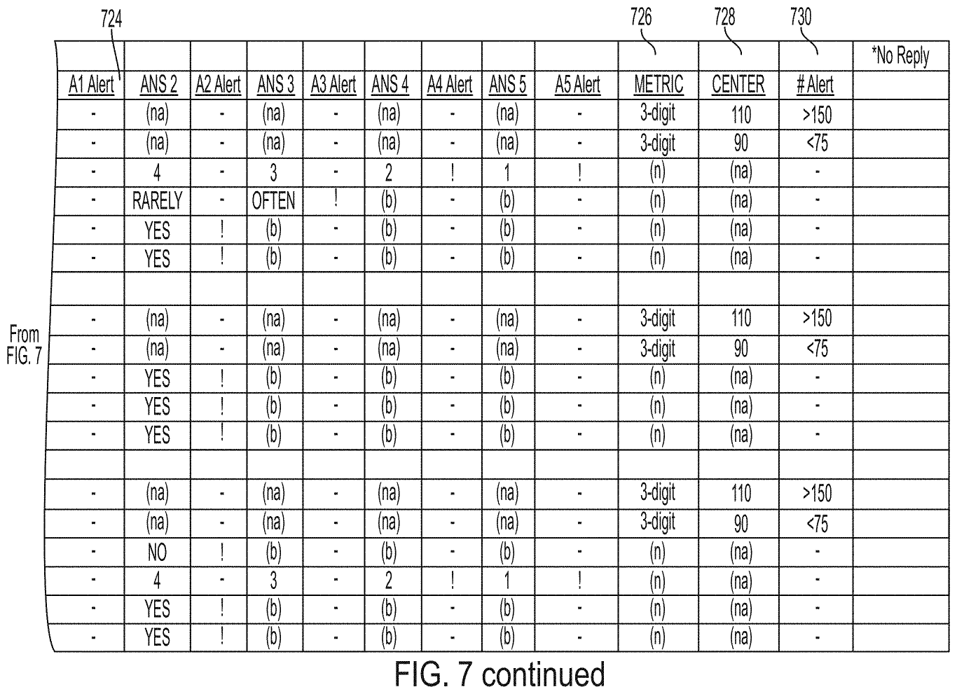

FIG. 7 depicts an example data file 700 that defines the tags and levels of urgency. The parts of the data file include a category of question 710, the journey ID and form ID 712, a question number 714, a patient journey for that specific illness or condition 716 and questions associated with that journey 718. The data layouts may be shown horizontally or vertically 720 which allows viewing of individual answers to queries. The historical and current query response 722 and the determined alert 724 are shown, which in a historical context allows detection of trends. The data output of the metric 726 may be numerical, yes or no, and the like, a median query answer 728 for that patient or that condition and an alert threshold 730 which may be modified by the healthcare profession are shown.

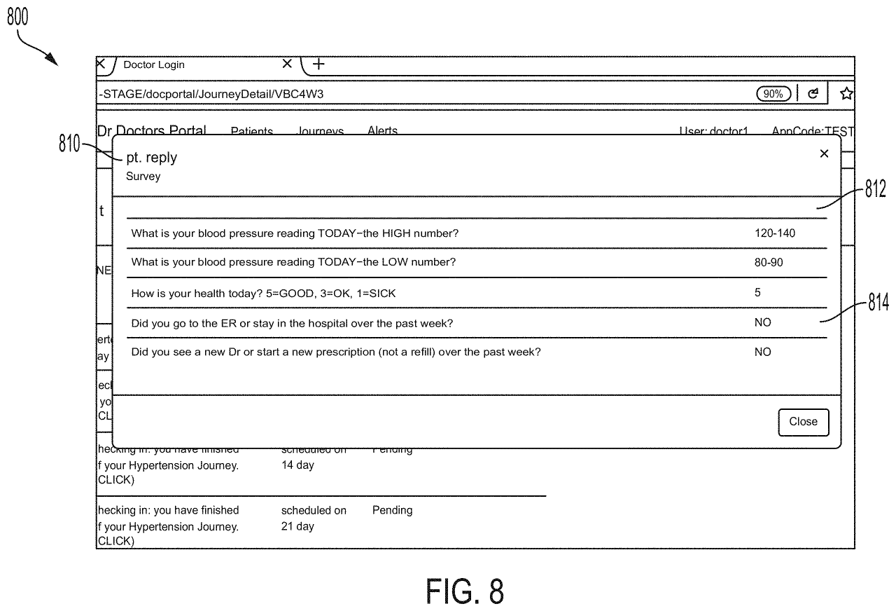

FIG. 8 depicts a patient survey reply 810 having a band of expected replies for blood pressure 812, how the patient feels and whether he went to the emergency room, hospital 814 or has started a new prescription.

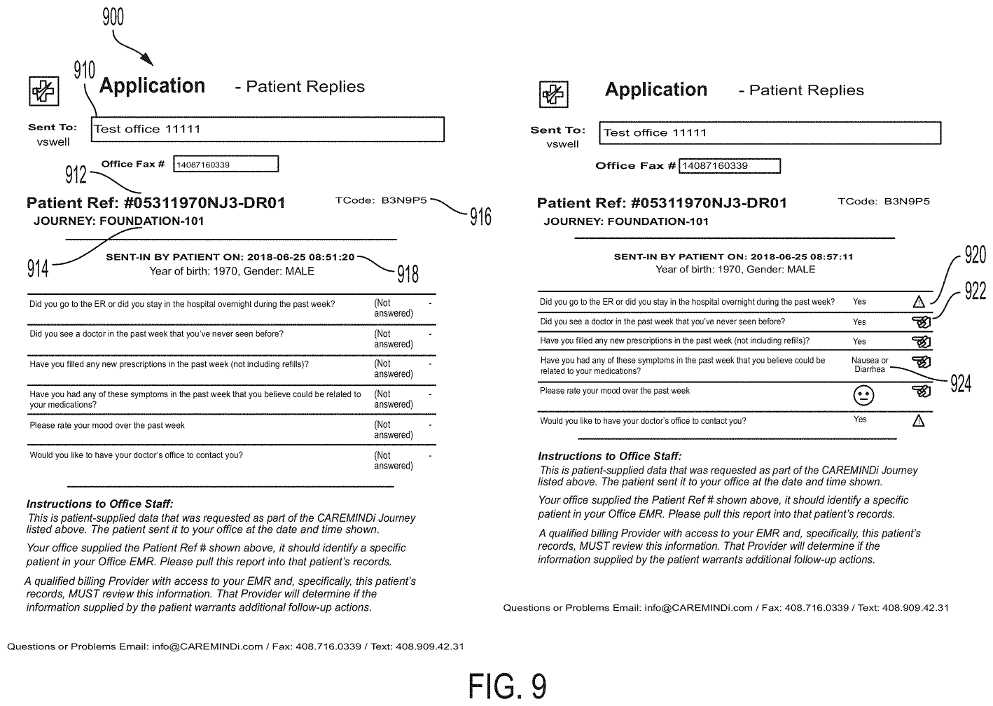

FIG. 9 depicts both a non-responsive and responsive reply set 900. The recipient 910, patient reference number 912, journey ID 914, unique T-Code 916 and timestamp 918 are depicted in the reply. A responsive report having alerts indicates an urgent issue 920, a follow up item 922 and an emoticon 924 indicating a patient feeling is shown.

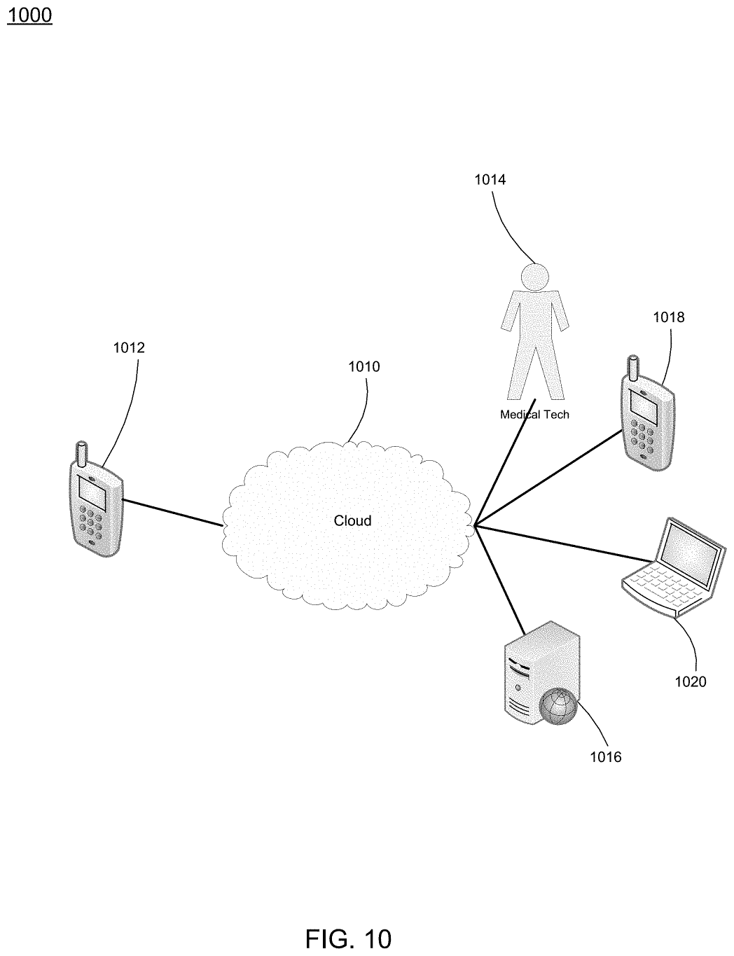

FIG. 10 depicts an example 1000 of communication associated with the alert tag. The cloud based system 1010 sends a request with queries to the patient's communication device 1012 which the patient fills out and returns. The cloud based system 1010 reviews the response and determines whether there are urgent or emergency issues and sends an urgent tagged response to the healthcare provider device.

If there is an emergency issue the cloud based system may contact or place the patient in contact with a medical technician 1014 in addition to notifying the healthcare provider by means of the healthcare provider's server 1016, the cloud based system may issue a text or message to the healthcare provider device. The communication route from the healthcare provider may be by means of mobile device 1018, computer 1020 or the like. The cloud based system may directly connect the patient via to the patient's communication device 1012 to the healthcare provider under appropriate circumstances. Non-urgent issues are sent to the healthcare provider device for later review.

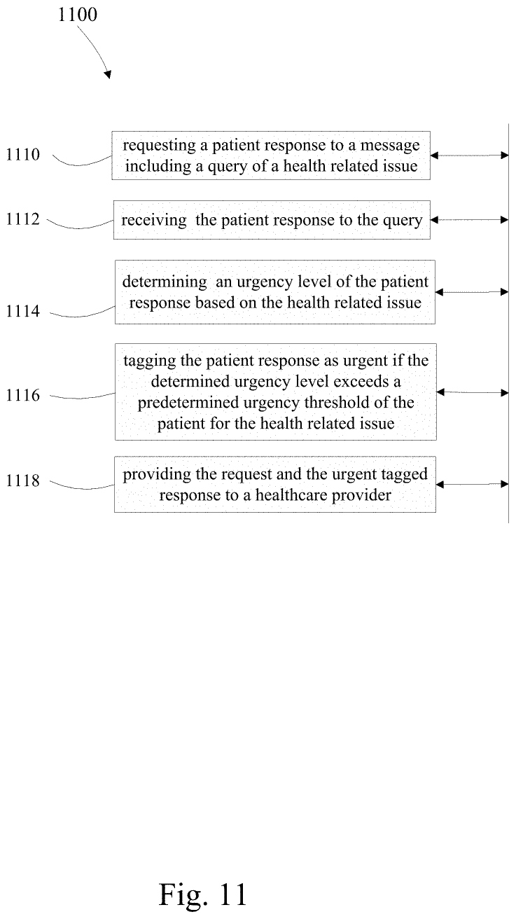

A second example method is shown in FIG. 11 related to response tagging, may include requesting 1110 a patient response to a message including a query of a health related issue and receiving 1112 the patient response to the query. The method then provides determining 1114 an urgency level of the patient response based on the health related issue, tagging 1116 the patient response as urgent if the determined urgency level exceeds a predetermined urgency threshold of the patient for the health related issue and providing 1118 the request and the urgent tagged response to a healthcare provider device. The method may also include proposing a set of work flow instructions linked to an urgent or non-urgent tagged response and presenting a clinical escalation review advisory if the response is tagged as urgent.

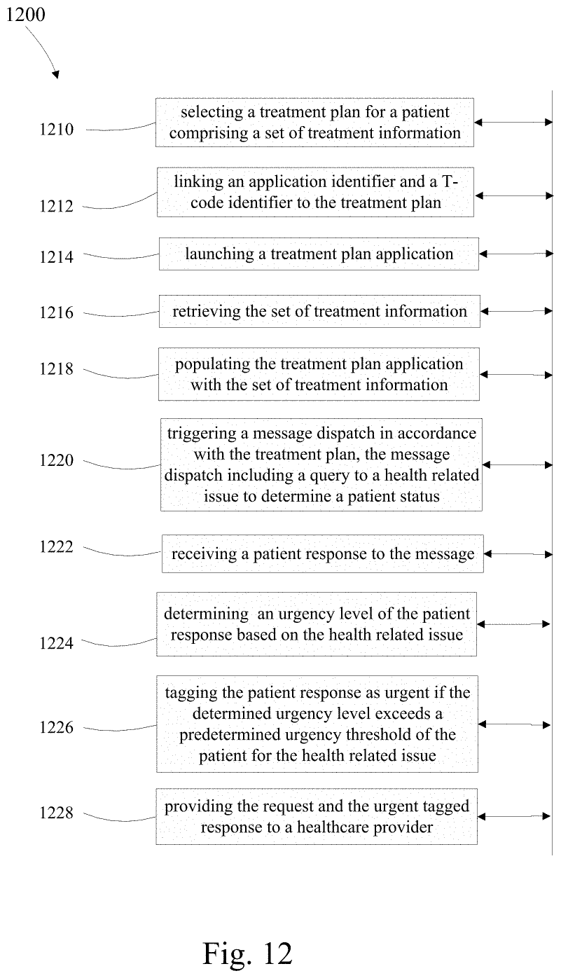

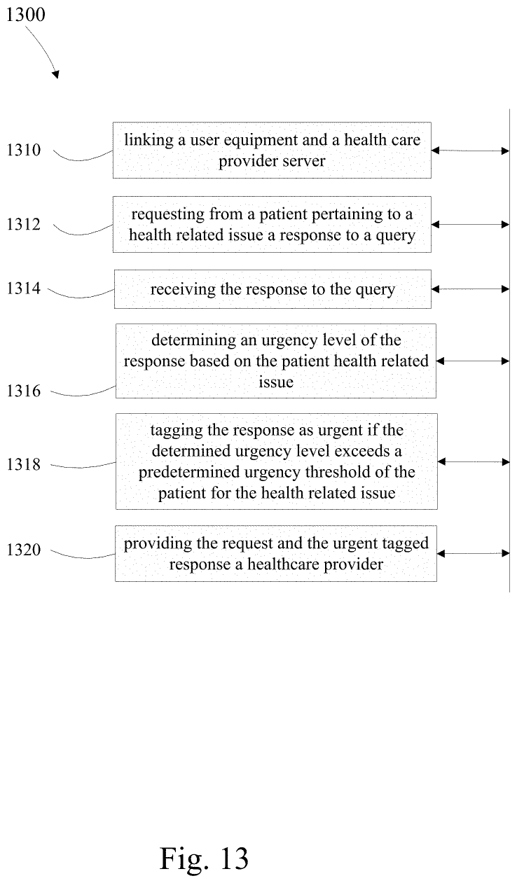

A first example method shown in FIG. 12, 1200, may include, selecting 1210 a treatment plan for a patient comprising a set of treatment information, linking 1212 an application identifier and a T-code identifier to the treatment plan and launching 1214 a treatment plan application. The method further includes retrieving 1216 the set of treatment information, populating 1218 the treatment plan application with the set of treatment information and triggering 1220 a message dispatch in accordance with the treatment plan. The message dispatch includes a query to a health related issue to determine a patient status and the system receives 1222 a patient response to the message. The method then provides determining 1224 an urgency level of the patient response based on the health related issue, tagging 1226 the patient response as urgent if the determined urgency level exceeds a predetermined urgency threshold of the patient for the health related issue and providing 1228 the request and the urgent tagged response to a healthcare provider device. The method may also include proposing a set of work flow instructions linked to an urgent or non-urgent tagged response and presenting a clinical escalation review advisory if the response is tagged as urgent.

With respect to the timing of patient responses, the first example method may also include, awaiting the patient response to the message for a late response duration and categorizing the patient response if the patient response is received within the late response duration. If the patient response is not received within the late response duration the method further comprises sending a duplicate message and flagging the patient response as non-responsive if the patient response to the duplicate message is not received within a second late response duration.

The timing of the message dispatches associated with the treatment plan is partly governed by a trigger table. The method may include loading the trigger table having a set of trigger dates based on the treatment plan where the message dispatch is sent according to the set of trigger dates. The method may further include receiving a message start date and receiving an initialization message from a patient mobile device to initiate the treatment plan and to initialize the set of trigger dates in the trigger table.

A first example non-transitory computer readable medium 1300 comprising instructions associated with the tagging of responses that, when read by a processor, cause the processor to perform; linking 1310 a patient device and a healthcare provider server, requesting 1312 from a patient pertaining to a health related issue a response to a query and receiving 1314 the response to the query. The processor then determines 1316 an urgency level of the response based on the patient health related issue, tags 1318 the response as urgent if the determined urgency level exceeds a predetermined urgency threshold of the patient for the health related issue and provides 1320 the request and the urgent tagged response a healthcare provider device.

The operations of a method or algorithm described in connection with the embodiments disclosed herein may be embodied directly in hardware, in a computer program executed by a processor, or in a combination of the two. A computer program may be embodied on a computer readable medium, such as a storage medium. For example, a computer program may reside in random access memory (“RAM”), flash memory, read-only memory (“ROM”), erasable programmable read-only memory (“EPROM”), electrically erasable programmable read-only memory (“EEPROM”), registers, hard disk, a removable disk, a compact disk read-only memory (“CD-ROM”), or any other form of storage medium known in the art.

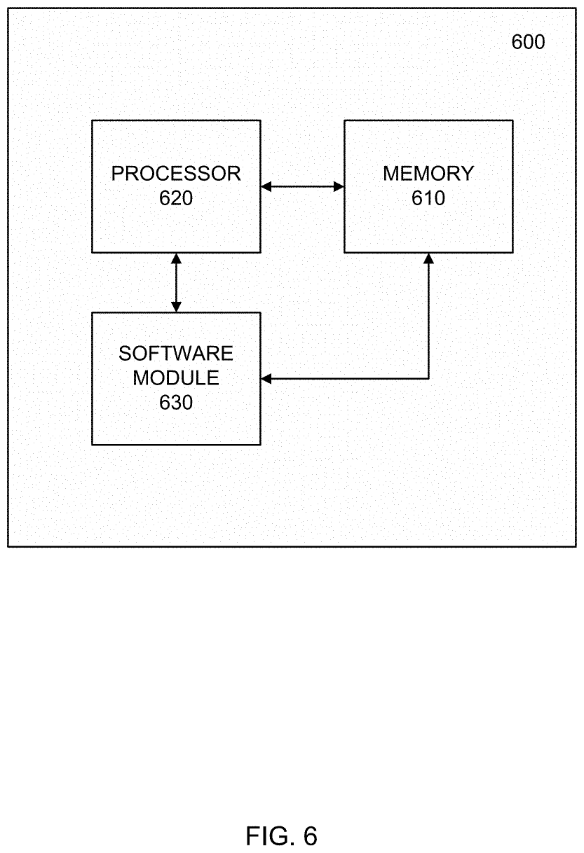

An exemplary storage medium may be coupled to the processor such that the processor may read information from, and write information to, the storage medium. In the alternative, the storage medium may be integral to the processor. The processor and the storage medium may reside in an application specific integrated circuit (“ASIC”). In the alternative, the processor and the storage medium may reside as discrete components. For example, FIG. 6 illustrates an example network element, which may represent any of the above-described network components of the other figures.

As illustrated in FIG. 6, a memory 610 and a processor 620 may be discrete components of the network entity 600 that are used to execute an application or set of operations. The application may be coded in software in a computer language understood by the processor 620, and stored in a computer readable medium, such as, the memory 610. The computer readable medium may be a non-transitory computer readable medium that includes tangible hardware components in addition to software stored in memory. Furthermore, a software module 630 may be another discrete entity that is part of the network entity 600, and which contains software instructions that may be executed by the processor 620. In addition to the above noted components of the network entity 600, the network entity 600 may also have a transmitter and receiver pair configured to receive and transmit communication signals (not shown).

Adjustable Guardrails

Flags set outer limits for biometric data, when a flag is reached or exceeded an urgency threshold may have been met that requires expedited attention from a healthcare provider. Within the flag outer limits are lower urgency boundaries referred to as guardrails. Analogously, an automobile will interact with a guardrail before breaching the safety of the highway and falling off of a cliff. The guardrails are cautions that precede the flags.

Flags may be set at points that are considered medically concerning in general, whereas guardrails may be more patient sensitive, and set at points that may present a caution to the healthcare provider for that patient before a situation becomes urgent.

This is a patient-facing portion of the program that is part of a system that automatically solicits health information from the patient, then delivers that information to that patient's healthcare provider device. The information that is requested is in a structured format that comprises a data set of responses.

The requests for information occur at repeated clinically relevant intervals, such as daily, across an extended time period. At a pre-established periodic time period such as weekly or bi-weekly the gathered information is forwarded to the patient's healthcare provider device. The provider is thereby able to monitor and assess the health status and trends of that patient.

As the daily information is gathered it is compared to guardrails which assesses whether the data being reported is within expected norms. These expected values are set specific to each patient. If the patient response data is outside of expected norms such as a biometric value exceeds a guardrail, the not yet reported information is immediately sent to the healthcare provider device.

This enables monitoring that can, for example, record daily information, but not be burdensome for the healthcare provider to review if it is within expected values. If a specific day's information is alarming such as exceeding a guardrail, or if a pattern trend over several days is alarming, then the information is immediately brought to the attention of the provider.

From time to time the guardrails may be reset to different values, to remain relevant to the patient's health status as it changes over time such as the patient is losing weight or gaining strength.

The approach or triggering of the guardrail alarm may find its root in a regimen that is ineffective for that particular patient. The system may assess the original treatment in light of a pool of similar patients to the current patient, based on condition, age, sex, demographics and the like and may propose an alternative treatment based on a positive outcome for the similar pool of patients. The treatments for the pool of patients and their outcomes may be stored on the cloud server in a format which does not identify the particular patients of the pool, but does describe their characteristics and outcomes. The data from the cloud server may be scrubbed and may be disease, patient characteristic, regimen and outcome centric. This collection and correlation of data may allow for continuous improvement in treatment proposals to the healthcare provider.

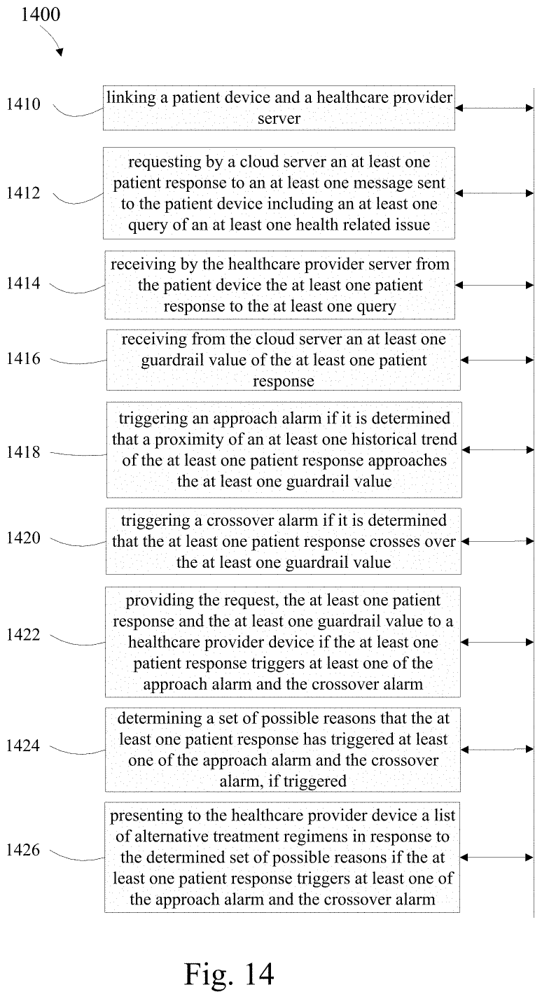

In one example depicted in FIG. 14 a method may comprise linking 1410 a patient device and a healthcare provider server, requesting 1412 by a cloud server an at least one patient response to an at least one message sent to the patient device including an at least one query of an at least one health related issue. The method also includes receiving 1414 by the healthcare provider server from the patient device the at least one patient response to the at least one query and receiving 1416 from the cloud server an at least one guardrail value of the at least one patient response. An approach alarm may be triggered 1418 if it is determined that proximity of a historical trend of the patient response approaches the guardrail value and a crossover alarm may be triggered 1420 if it is determined that the patient response crosses over the guardrail value. If the patient response triggers either the approach alarm or the crossover alarm, the request and the patient response and the guardrail value are sent 1422 to a healthcare provider device. The method also determines 1424 a set of possible reasons that the at least one patient response has triggered at least one of the approach alarm and the crossover alarm, if triggered and presents 1426 to the healthcare provider device a list of alternative treatment regimens in response to the determined set of possible reasons if the at least one patient response triggers at least one of the approach alarm and the crossover alarm.

Anytime there is reference to a patient response herein, it is equivalent to stating the response is from a patient device (which may be a wired and/or wireless device).

The cloud server may receive the patient responses and forward them to the healthcare provider server. The cloud server may keep track of responses, patient demographics, age, sex and the like and may track how the patient is responding to the regimen. The cloud server may also keep a database of patient characteristics in a pool and healthcare regimens and track how portions of the pool respond to the various treatments. In this way, it is possible for the cloud server to find possible alternative treatments that apply to specific sub-groups within the patient pool that respond most favorably to specific treatments. The pools may be stripped of data that may identify specific patients to insure patient confidentiality.

The historical trend may be determined over a predetermined period, such as two or three days or a longer average such as a week or more, also the historical trend may be based on a moving average, a rolling average, a weighted moving average and the like. The guardrail value may be reset based on an updated patient health status. An urgency level of the patient response may be determined, and the patient response may be tagged as urgent if a determined urgency level exceeds a predetermined urgency threshold. The system may provide the healthcare provider with its reason for providing the request, the patient response and the guardrail value and the system may also propose a set of workflow instructions linked to the provided reason. Additionally, the system may display at the wireless device the historical trend and send a follow-on query to a patient.

In another example, a non-transitory computer readable medium comprises instructions that, when read by a processor, cause the processor to perform operations such as requesting by a cloud server an at least one patient response to an at least one message sent to a patient device including an at least one query of an at least one health related issue, receiving by a healthcare provider server the at least one patient response from the patient device to the at least one query and receiving from the cloud server an at least one guardrail value of the at least one patient response. The instructions include instructions causing the processor to trigger an approach alarm if it is determined that a proximity of an at least one historical trend of the at least one patient response approaches the at least one guardrail value, trigger a crossover alarm if it is determined that the at least one patient response crosses over the at least one guardrail value and providing the request, the at least one patient response and the at least one guardrail value to a healthcare provider device if the at least one patient response triggers at least one of the approach alarm and the crossover alarm. The instructions also include instructions causing the processor to determine a set of possible reasons that the at least one patient response triggered at least one of the approach alarm and the crossover alarm, if triggered and present to the healthcare provider device a list of alternative treatment regimens in response to the determined set of possible reasons if the at least one patient response triggers at least one of the approach alarm and the crossover alarm.

In yet a further example, a system comprises an at least one cloud-based processor and at least one memory electrically coupled to the at least one cloud-based processor and storing an application. The at least one cloud-based processor performs operations to request by a cloud server an at least one patient response to an at least one message sent to a patient device including an at least one query of an at least one health related issue, receive by a healthcare provider server the at least one patient response from the patient device to the at least one query and receive from the cloud server an at least one guardrail value of the at least one patient response. The at least one cloud-based processor further performs operations to trigger an approach alarm if it is determined that a proximity of an at least one historical trend of the at least one patient response approaches the at least one guardrail value, trigger a crossover alarm if it is determined that the at least one patient response crosses over the at least one guardrail value and provide by the cloud server, the request, the at least one patient response and the at least one guardrail value from the cloud server to a healthcare provider device if the at least one patient response triggers at least one of the approach alarm and the crossover alarm. The at least one cloud-based processor also performs operations to determine a set of possible reasons that the at least one patient response triggered at least one of the approach alarm and the crossover alarm, if triggered and present to the healthcare provider device a list of alternative treatment regimens in response to the determined set of possible reasons if the at least one patient response triggers at least one of the approach alarm and the crossover alarm.

Progressive Improvement Thresholding

Biometric measurement data from a patient varies from measurement to measurement. Over a set of measurements, the set has a mean or average and a standard deviation. The standard deviation may be normal in that it is equal on an upper side and a lower side, or may have an offset called a kurtosis that is larger on one side than the other. This set of measurements and the historical statistical data indicates a patient's biometric response to a treatment plan.

If the healthcare professional deems the historical statistical data as a baseline for the patient, any meaningful change in the statistics or trend from the baseline indicates a change either for increased health and wellness or decreased health or wellness.

Given the patient baseline, the statistical output has a mean and standard deviation boundaries. It is possible to set low urgency thresholds at one of the boundaries to determine the efficacy of a treatment regimen.

Patients have differing responses to new treatments or changes in treatments. These differing responses may indicate either provide positive progress or in the negative indicate a failure to progress. Patient responses whether subtle progression, profound progression or failure to progress may be measured statistically against the baseline.

Within the concept of adjustable guardrails there exist low urgency thresholds which have effects that are not immediately urgent and high urgency thresholds which are immediately urgent. The low urgency thresholds and the patient's responses within that band may provide vital clues as to treatments effectiveness.

As an example, if blood glucose is measured daily, and a historical background has been established, it is possible to set low urgency thresholds so that half of the readings are above the low urgency threshold and half the readings are below the low urgency threshold. In effect, the low urgency threshold would be set to the historical mean. If a change in medication, or medication dosage is administered to the patient, a change in the number of readings above and below low urgency threshold may result. If there was no change to the number of readings above and below the low urgency threshold result, or if the readings indicate a worsening result, then the outcome is deemed a failure to progress, or FTP.

The healthcare professional may set the low urgency threshold slightly above the historical mean, so that if the number of points that crosses the threshold increases, an short-term issue may be immediately detected and dealt with. If on the other hand the number of threshold violations decreases then the short term progress is positive. At this point the low urgency threshold may be further modified to determine the extent of the progress.

Healthcare professionals are most concerned that the treatments do no harm. So that a failure to progress situation begins with an assessment of the current state of the patient, and after the treatment modification, an assessment is made whether the modification made the patient progress and pull away from the low urgency threshold.

The assessment of a failure to progress may be detected by counting the percentage of points on a failure side of the threshold. If the percentage of points on the failure side either stays the same or increases, a failure to progress is indicated.

Changes to the treatment plan and a resulting success would allow the healthcare professional to progressively move the low urgency threshold down and reassess treatment options for an optimal patient outcome.

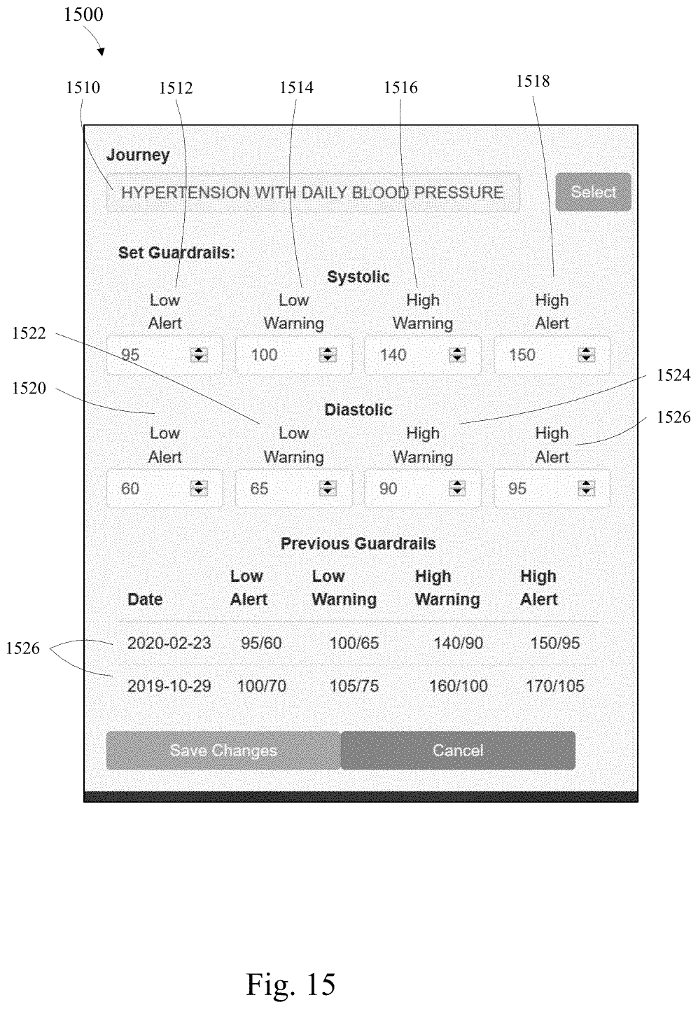

An example control panel programming input for guardrail warning and alert levels is depicted in FIG. 15. In this example, the journey title 1510 is selected for a hypertensive patient and preset guardrails are input which may be adjusted by the healthcare provider for systolic guardrails for low alert 1512, low warning 1514, high warning 1516 and high alert 1518. Additionally, the diastolic low alert 1520, low warning 1522, high warning 1524 and high alert 1526. In this example, the good level for systolic is between 100 and 140 mm-hg and between 65 and 90 mm-hg for diastolic. Going past those guardrails triggers either a warning or an alert. Previous guardrails are shown as 1526 and the date and low alert, low warning, high warning and high alert for systolic and diastolic blood pressure readings. In this way the dates and times for the guardrail values are historically saved and allows the healthcare provider to see what previous guardrail values were. The programming screen is displayed for the healthcare provider via the cloud server and the guardrails may be stored within the cloud server. The values and dates of the guardrails and changes may additionally be stored in the healthcare provider server.

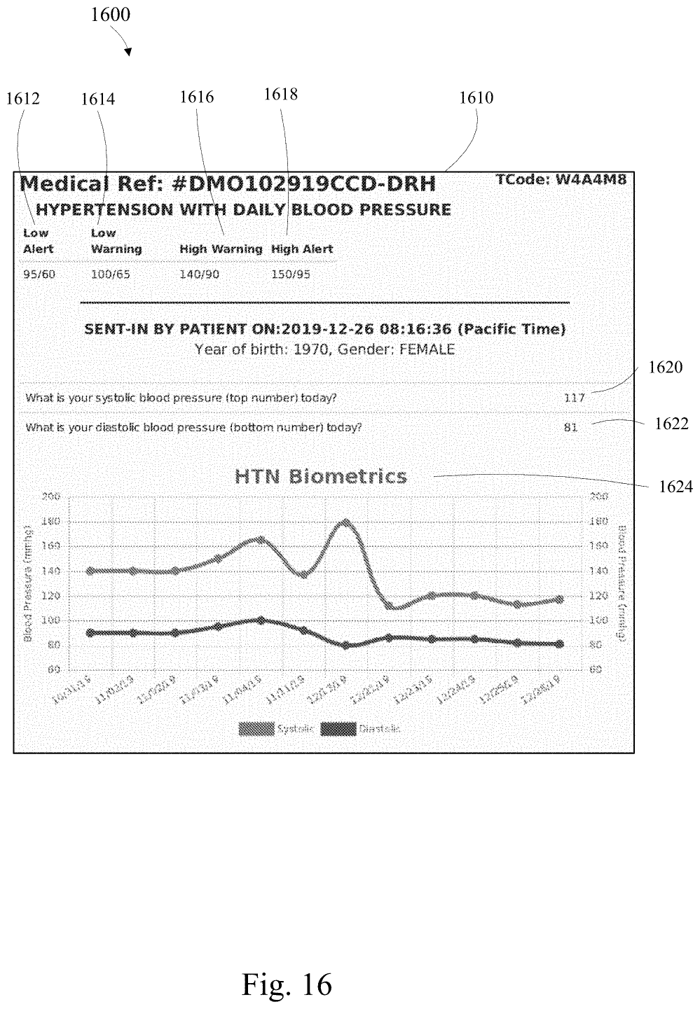

An example patient report called an encounter summary is depicted in FIG. 16. In this example the patient chart has the medical reference number 1610, and the systolic and diastolic guardrails for low alert 1612, low warning 1614, high warning 1616 and high alert 1618. This encounter summary shows the history for the hypertensive patient with the guardrails shown at the top of the summary. The current systolic 1620 and diastolic 1622 values are shown on the screen. The historical systolic and diastolic metrics are shown in 1624. This example encounter summary shows an overall progression of the patient's blood pressure during the historical timeframe shown. The data may be displayed for the healthcare provider and the data may be stored in both the cloud server and the healthcare provider server.

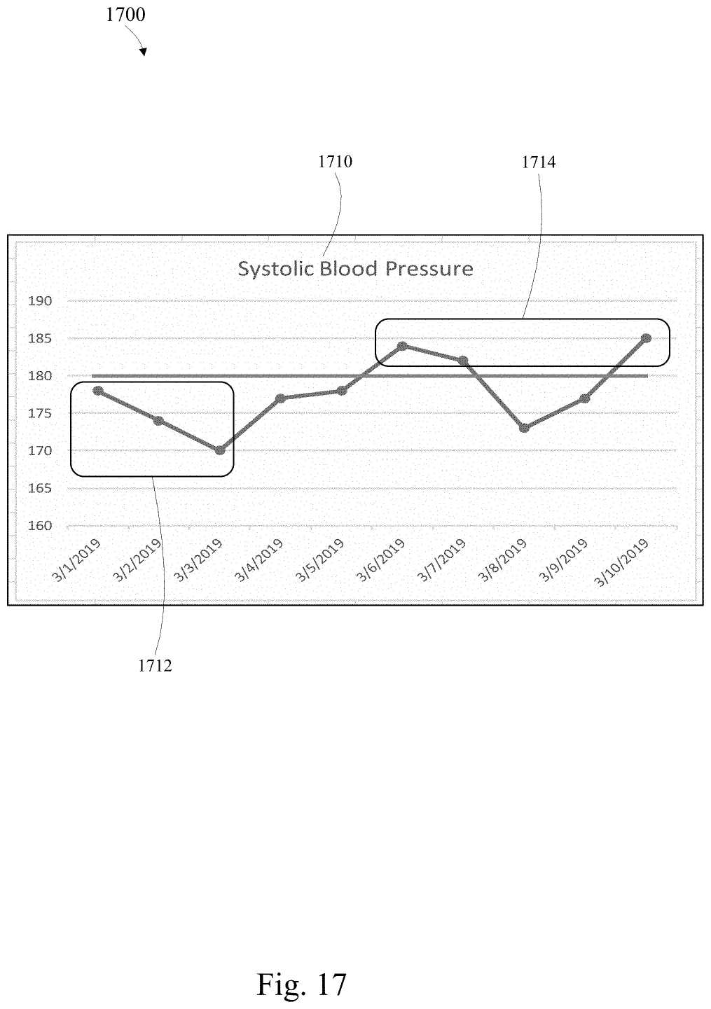

An example failure to progress chart for systolic blood pressure is depicted in FIG. 17. In this example chart the systolic blood pressure trend 1710 is depicted. The first three values shown as 1712 appear to be improving for the patient with the first five values below the 180 mm-hg value. The high warning value of 180 mm-hg of systolic pressure sets the bar against which the data is reviewed. The three data points 1714 exceed the warning value, and tend to indicate that the patient is experiencing a failure to progress. The failure may be one of not adhering to the drug schedule, stress, high salt foods etc. One-time events may not indicate a failure to progress, however, multiple events over a specific timeframe may indicate a failure to progress. The healthcare provider may determine the number of warnings within a specific time frame to indicate a failure to progress, or, a lack of reduction of the trend of the measurements. In the case there where progress is seen, the high warning value may be reduced as determined by the healthcare provider or suggested by the cloud based server.

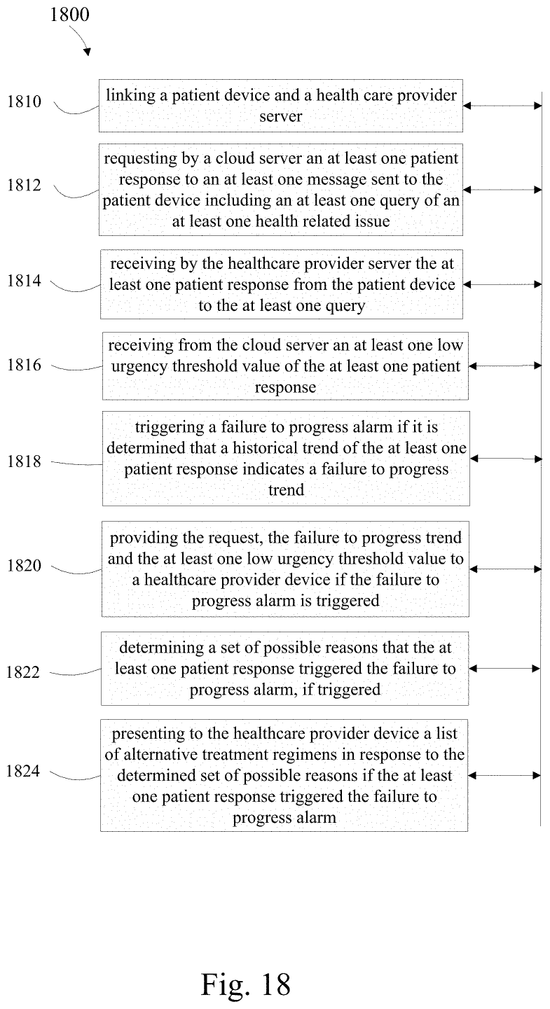

An example method for detecting a failure to progress is depicted in FIG. 18, comprising linking 1810 a patient device and a healthcare provider server and requesting 1812 by a cloud server an at least one patient response to an at least one message sent to the patient device including an at least one query of an at least one health related issue. The method further includes receiving 1814 by the healthcare provider server the at least one patient response from the patient device to the at least one query and receiving 1816 from the cloud server an at least one low urgency threshold value of the at least one patient response. The method also includes triggering 1818 a failure to progress alarm if it is determined that a historical trend of the at least one patient response indicates a failure to progress trend and providing 1820 the request, the failure to progress trend and the at least one low urgency threshold value to a healthcare provider device if the failure to progress alarm is triggered. The method includes determining 1822 a set of possible reasons that the at least one patient response triggered the failure to progress alarm, if triggered and presenting 1824 to the healthcare provider device a list of alternative treatment regimens in response to the determined set of possible reasons if the at least one patient response triggered the failure to progress alarm.

Although an exemplary embodiment of the system, method, and computer readable medium of the present application has been illustrated in the accompanied drawings and described in the foregoing detailed description, it will be understood that the application is not limited to the embodiments disclosed, but is capable of numerous rearrangements, modifications, and substitutions without departing from the spirit or scope of the application as set forth and defined by the following claims. For example, the capabilities of the system of the various figures can be performed by one or more of the modules or components described herein or in a distributed architecture and may include a transmitter, receiver or pair of both. For example, all or part of the functionality performed by the individual modules, may be performed by one or more of these modules. Further, the functionality described herein may be performed at various times and in relation to various events, internal or external to the modules or components. Also, the information sent between various modules can be sent between the modules via at least one of: a data network, the Internet, a voice network, an Internet Protocol network, a wireless device, a wired device and/or via plurality of protocols. Also, the messages sent or received by any of the modules may be sent or received directly and/or via one or more of the other modules.

One skilled in the art will appreciate that a “system” could be embodied as a personal computer, a server, a console, a personal digital assistant (PDA), a cell phone, a tablet computing device, a smartphone or any other suitable computing device, or combination of devices. Presenting the above-described functions as being performed by a “system” is not intended to limit the scope of the present application in any way, but is intended to provide one example of many embodiments of the present application. Indeed, methods, systems and apparatuses disclosed herein may be implemented in localized and distributed forms consistent with computing technology.

It should be noted that some of the system features described in this specification have been presented as modules, in order to more particularly emphasize their implementation independence. For example, a module may be implemented as a hardware circuit comprising custom very large scale integration (VLSI) circuits or gate arrays, off-the-shelf semiconductors such as logic chips, transistors, or other discrete components. A module may also be implemented in programmable hardware devices such as field programmable gate arrays, programmable array logic, programmable logic devices, graphics processing units, or the like.

A module may also be at least partially implemented in software for execution by various types of processors. An identified unit of executable code may, for instance, comprise one or more physical or logical blocks of computer instructions that may, for instance, be organized as an object, procedure, or function. Nevertheless, the executables of an identified module need not be physically located together, but may comprise disparate instructions stored in different locations which, when joined logically together, comprise the module and achieve the stated purpose for the module. Further, modules may be stored on a computer-readable medium, which may be, for instance, a hard disk drive, flash device, random access memory (RAM), tape, or any other such medium used to store data.

Indeed, a module of executable code could be a single instruction, or many instructions, and may even be distributed over several different code segments, among different programs, and across several memory devices. Similarly, operational data may be identified and illustrated herein within modules, and may be embodied in any suitable form and organized within any suitable type of data structure. The operational data may be collected as a single data set, or may be distributed over different locations including over different storage devices, and may exist, at least partially, merely as electronic signals on a system or network.

It will be readily understood that the components of the application, as generally described and illustrated in the figures herein, may be arranged and designed in a wide variety of different configurations. Thus, the detailed description of the embodiments is not intended to limit the scope of the application as claimed, but is merely representative of selected embodiments of the application.

One having ordinary skill in the art will readily understand that the application as discussed above may be practiced with steps in a different order, and/or with hardware elements in configurations that are different than those which are disclosed. Therefore, although the application has been described based upon these preferred embodiments, it would be apparent to those of skill in the art that certain modifications, variations, and alternative constructions would be apparent, while remaining within the spirit and scope of the application. In order to determine the metes and bounds of the application, therefore, reference should be made to the appended claims.

While preferred embodiments of the present application have been described, it is to be understood that the embodiments described are illustrative only and the scope of the application is to be defined solely by the appended claims when considered with a full range of equivalents and modifications (e.g., protocols, hardware devices, software platforms etc.) thereto.

What is claimed is:

1. A method, comprising:
linking a patient device and a healthcare provider server;
requesting by a cloud server an at least one patient response to an at least one message sent to the patient device including an at least one query of an at least one health related issue;
receiving by the healthcare provider server from the patient device the at least one patient response to the at least one query;
receiving from the cloud server an at least one guardrail value of the at least one patient response;
triggering an approach alarm if it is determined that a proximity of an at least one historical trend of the at least one patient response approaches the at least one guardrail value;
triggering a crossover alarm if it is determined that the at least one patient response crosses over the at least one guardrail value;
providing the request, the at least one patient response and the at least one guardrail value to a healthcare provider device if the at least one patient response triggers at least one of the approach alarm and the crossover alarm;
determining a set of possible reasons that the at least one patient response has triggered at least one of the approach alarm and the crossover alarm, if triggered; and
presenting to the healthcare provider device a list of alternative treatment regimens in response to the determined set of possible reasons if the at least one patient response triggers at least one of the approach alarm and the crossover alarm.

2. The method of claim 1, wherein the at least one historical trend is determined over a predetermined period.

3. The method of claim 1, wherein the at least one historical trend is based on at least one of a moving average, a rolling average and a weighted moving average.

4. The method of claim 1, further comprising retriggering the at least one guardrail value based on an updated patient health status.

5. The method of claim 1, further comprising determining an urgency level of the at least one patient response.

6. The method of claim 5, further comprising tagging the at least one patient response as urgent if a determined urgency level exceeds a predetermined urgency threshold.

7. The method of claim 1, further comprising providing a reason for providing the request, the at least one patient response and the guardrail value to the healthcare provider device.

8. The method of claim 7, further comprising proposing a set of workflow instructions linked to the provided reason.

9. The method of claim 1, further comprising displaying at the patient device the at least one historical trend.

10. The method of claim 1, further comprising sending by the cloud server an at least one follow-on query to the patient device.

11. A non-transitory computer readable medium comprising instructions that, when read by a processor, cause the processor to perform:
requesting by a cloud server an at least one patient response to an at least one message sent to a patient device including an at least one query of an at least one health related issue;
receiving by a healthcare provider server the at least one patient response from the patient device to the at least one query;
receiving from the cloud server an at least one guardrail value of the at least one patient response;
triggering an approach alarm if it is determined that a proximity of an at least one historical trend of the at least one patient response approaches the at least one guardrail value;
triggering a crossover alarm if it is determined that the at least one patient response crosses over the at least one guardrail value;
providing the request, the at least one patient response and the at least one guardrail value to a healthcare provider device if the at least one patient response triggers at least one of the approach alarm and the crossover alarm;
determining a set of possible reasons that the at least one patient response triggered at least one of the approach alarm and the crossover alarm, if triggered; and
presenting to the healthcare provider device a list of alternative treatment regimens in response to the determined set of possible reasons if the at least one patient response triggers at least one of the approach alarm and the crossover alarm.

12. The non-transitory computer readable medium of claim 11, wherein the at least one historical trend is determined over a predetermined period.

13. The non-transitory computer readable medium of claim 11, wherein the at least one historical trend is based on at least one of a moving average, a rolling average and a weighted moving average.

14. The non-transitory computer readable medium of claim 11, comprising instructions, that when read by a processor, cause the processor to perform retriggering the at least one guardrail value based on an updated patient health status.

15. The non-transitory computer readable medium of claim 11, comprising instructions, that when read by a processor, cause the processor to perform determining an urgency level of the patient response.

16. The non-transitory computer readable medium of claim 15, comprising instructions, that when read by a processor, cause the processor to perform tagging the patient response as urgent if a determined urgency level exceeds a predetermined urgency threshold.

17. The non-transitory computer readable medium of claim 11, comprising instructions, that when read by a processor, cause the processor to perform providing a reason for providing the request, the patient response and the guardrail value to the healthcare provider device.

18. The non-transitory computer readable medium of claim 17, comprising instructions, that when read by a processor, cause the processor to perform proposing a set of work flow instructions linked to the provided reason.

19. A system, comprising:
an at least one cloud-based processor; and
at least one memory electrically coupled to the at least one cloud-based processor and storing an application, wherein the at least one cloud-based processor performs operations to:
request by a cloud server an at least one patient response to an at least one message sent to a patient device including an at least one query of an at least one health related issue;
receive by a healthcare provider server the at least one patient response from the patient device to the at least one query;
receive from the cloud server an at least one guardrail value of the at least one patient response;
trigger an approach alarm if it is determined that a proximity of an at least one historical trend of the at least one patient response approaches the at least one guardrail value;
trigger a crossover alarm if it is determined that the at least one patient response crosses over the at least one guardrail value;
provide by the cloud server, the request, the at least one patient response and the at least one guardrail value from the cloud server to a healthcare provider device if the at least one patient response triggers at least one of the approach alarm and the crossover alarm;
determine a set of possible reasons that the at least one patient response triggered at least one of the approach alarm and the crossover alarm, if triggered; and
present to the healthcare provider device a list of alternative treatment regimens in response to the determined set of possible reasons if the at least one patient response triggers at least one of the approach alarm and the crossover alarm.

20. A method, comprising:
linking a patient device and a healthcare provider server;
requesting by a cloud server an at least one patient response to an at least one message sent to the patient device including an at least one query of an at least one health related issue;
receiving by the healthcare provider server the at least one patient response from the patient device to the at least one query;
receiving from the cloud server an at least one low urgency threshold value of the at least one patient response;
triggering a failure to progress alarm if it is determined that a historical trend of the at least one patient response indicates a failure to progress trend;
providing the request, the failure to progress trend and the at least one low urgency threshold value to a healthcare provider device if the failure to progress alarm is triggered;
determining a set of possible reasons that the at least one patient response triggered the failure to progress alarm, if triggered; and
presenting to the healthcare provider device a list of alternative treatment regimens in response to the determined set of possible reasons if the at least one patient response triggered the failure to progress alarm.
